(19) United States
(12) Patent Application Publication
(10) Pub. No.: US 2003/0078433 A1
(43) Pub. Date: Apr. 24, 2003

(54) Process for preparing 4,5-dihydro-1,3-thiazoles

(76) Inventors: Katrin Joschek, Koln (DE); Anton Vidal-Ferran, Koln (DE); Manfred Jautelat, Burscheid (DE); Michael Schelhaas, Koln (DE)

Correspondence Address: BAYER CORPORATION, PATENT DEPARTMENT, 100 BAYER ROAD, PITTSBURGH, PA, 15205, US

(21) Appl. No.: 10/229,833

(22) Filed: Aug. 28, 2002

(30) Foreign Application Priority Data
Aug. 31, 2001 (DE) 10142749.2

(51) Int. Cl. C07D 277/18

(52) U.S. Cl. 548/200

(57) ABSTRACT

The present invention relates to an improved and more economical process for the synthesis of 4,5-dihydro-1,3-thiazoles carried out in a single vessel without the isolation of intermediates.

### BACKGROUND OF THE INVENTION

The present invention relates to an improved and at the same time more economical process for the synthesis of 4,5-dihydro-1,3-thiazoles.

4,5-Dihydro-1,3-thiazoles are materials that have been known for a long time and are used, inter alia, as key intermediates for the synthesis of dihydrothiazole- and thiazole-based active compounds in the agrochemical and pharmaceutical industries.

An efficient synthetic route giving very good selectivities and yields is required for the preparation of 4,5-dihydro-1,3-thiazoles. The starting materials needed for this purpose must be available on an industrial scale.

The class of 4,5-dihydro-1,3-thiazoles (I) is known and their synthesis is described, for example, in DE-A 1,964,276 and U.S. Pat. No. 3,678,064. In the synthetic route described, 1-amino-2-alkanethiols (II) are reacted with 2,2-dialkoxyalkanenitriles (III) to give the ketals (IV), which are converted by hydrolysis into the desired 4,5-dihydro-1,3-thiazoles (I). The preparation of 2,2-dialkoxyalkanenitriles (III) by the method of DE-A 1,964,276 requires an unacceptable reaction time of 40 days. An improved process described in Synthesis, 1983, 498-500, gives a yield of 83% by weight. The reaction time here is from 3 to 12 hours.

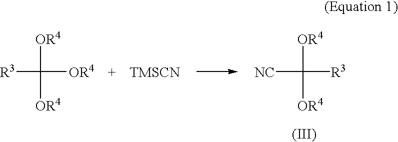

TMSCN=trimethylsilyl cyanide

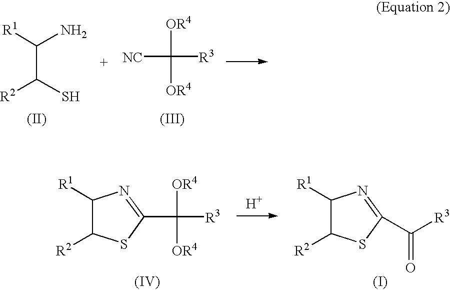

In the formulas of Equations 1 and 2, R¹, R², R³, and R⁴ are each, independently of one another, hydrogen or an organic radical having from 1 to 10 carbon atoms.

The first step in Equation 2 requires 1.57 equivalents of 1-amino-2-alkanethiol (II), which is very expensive. The disadvantage of the synthetic route described is the complicated work-up steps with isolation of the intermediates. With a view to industrial implementation, the hydrolysis using an excess of concentrated sulfuric acid (i.e., 15.5 equivalents) is particularly critical, since these large amounts of acid subsequently must be neutralized, which is highly exothermic. In addition, the neutralization forms a large quantity of salts, which is undesirable from an ecological point of view. Each step of the process described is followed by an aqueous work-up with subsequent purification. The aqueous work-up is always associated with formation of a considerable quantity of salts, which is likewise disadvantageous in an industrial process.

It was therefore an object of the invention to improve the process so that it can be implemented industrially while taking into account ecological aspects and so that the disadvantages of the earlier process are overcome. This object has been able to be achieved according to the invention.

It has surprisingly been found that the entire synthesis sequence can be carried out as a single-vessel synthesis without complicated work-up steps.

### SUMMARY OF THE INVENTION

The invention accordingly provides a process for preparing 4,5-dihydro-1,3-thiazoles of formula (I)

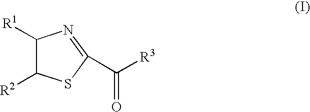

where R¹, R², and R³ are each, independently of one another,

hydrogen or an organic radical having from 1 to 10 carbon atoms, comprising

(1) reacting a trialkoxyalkane of the formula

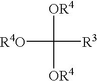

where R³ and R⁴ are each, independently of one another, hydrogen or an organic radical having from 1 to 10 carbon atoms,

with CN⁻ to form a 2,2-dialkoxyalkanenitrile of the formula

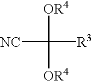

where R³ and R⁴ are defined as above,

(2) reacting the 2,2-dialkoxyalkanenitrile with an aminoalkanethiol of the formula

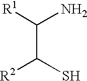

where R¹ and R² are defined as above, to form a ketal of the formula

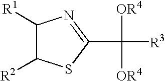

where R¹, R², R³, and R⁴ are defined as above, and

(3) hydrolyzing the ketal with an acid to form the 4,5-dihydro-1,3-thiazole of formula (I),

wherein the entire reaction sequence is carried out in a single vessel without isolation of intermediates.

The process of the invention can be summarized by the following reaction sequence:

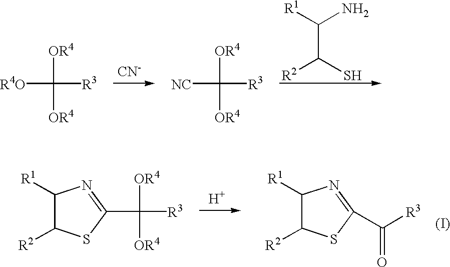

### DETAILED DESCRIPTION OF THE INVENTION

Examples of functional groups by which the organic radicals R¹, R², and R³ can be substituted are alcohols and halogens. R¹ and R² are preferably hydrogen or alkyl groups having from 1 to 10 carbon atoms and are particularly preferably each hydrogen. R³ is preferably an alkyl group having from 1 to 10 carbon atoms and is particularly preferably ethyl. R⁴ is preferably an alkyl group having from 1 to 10 carbon atoms and is particularly preferably methyl, ethyl, or propyl.

The overall yield in, for example, the synthesis of 2-propionyl-4,5-dihydro-1,3-thiazole (formula (I) in which R³ is C₂H₅) is 40%. In addition, the amount of 1-amino-2-alkanethiol (II) was able to be reduced from 1.57 equivalent to 1.1 equivalent.

TABLE
Comparison of the yields

| | Amount | Amount | | | | | Overall |
|---|---|---|---|---|---|---|---|
| | of (II) | of (III) | | | | | yield |
| Process | [mol] | [mol] | R¹ | R² | R³ | R⁴ | [%] |
| DE-A | 1.57 | 1.0 | H | H | C₂H₅ | C₂H₅ | 16 |
| 1,964,276 | | | | | | | |
| Example 3 | 1.10 | 1.0 | H | H | C₂H₅ | CH₃ | 40 |
| (according to | | | | | | | |
| the invention) | | | | | | | |

A first advantage of the invention is the significantly better technical manageability, since a number of work-up and purification steps can be saved due to the single-vessel synthesis. Secondly, the amount of acid required was able to be reduced from 15 equivalents to 5 equivalents, which is a great advantage, particularly with a view to an industrial synthesis. The amount of salts formed in the neutralization is greatly reduced as a result.

In the process of the invention for preparing 4,5-dihydro-1,3-thiazoles of the formula (I), preference is given to heating equimolar amounts of trialkoxyalkane and cyanide (preferably from trimethylsilyl cyanide) with addition of catalytic amounts of a Lewis acid (preferably ZnCl₂) in a temperature range from 40 to 100° C. (preferably in a temperature range from 55 to 70° C.) for from 3 to 20 hours (preferably for a time of from 12 to 18 hours). After cooling, from 1.0 to 1.5 equivalents (preferably from 1.0 to 1.2 equivalents) of 1-amino-2-alkanethiol (II) in an organic solvent are added. As organic solvent, preference is given to using polar solvents, e.g., alcohols. The reaction mixture is then heated to from 40 to 100° C.; the reaction temperature is preferably from 60 to 80° C. The reaction time is from 3 to 20 hours, preferably from 12 to 18 hours. The solvent is preferably distilled off under reduced pressure. From 5 to 30 equivalents (preferably 5 to 15 equivalents, particularly preferably 5 to 7 equivalents) of an acid (preferably concentrated sulfuric acid) are added dropwise to the remaining reaction mixture at a temperature of from 10° C. to −10° C. (preferably from 0° C. to 5° C.). After stirring at the above-mentioned temperature for from 1 to 5 hours (preferably from 1 to 3 hours), the reaction mixture is neutralized by means of an aqueous base (preferably NaHCO₃). After extraction of the 4,5-dihydro-1,3-thiazole (I) into an organic phase, preferably using dichloromethane or an organic ether (e.g., diethyl ether), as solvent, the desired compounds are isolated in a yield of about 40%.

The following examples further illustrate details for the process of this invention. The invention, which is set forth in the foregoing disclosure, is not to be limited either in spirit or scope by these examples. Those skilled in the art will readily understand that known variations of the conditions of the following procedures can be used. Unless otherwise noted, all temperatures are degrees Celsius and all percentages are percentages by weight.

### EXAMPLES

### Example 1

### 2-(1,1-Dimethoxypropyl)-4,5-dihydro-1,3-thiazole (IV)—Individual Synthesis Steps

Under argon, 20.04 g of anhydrous ammonium acetate (260 mmol), 6.79 g (88 mmol) of cysteamine, and 10.33 g (80 mmol) of 2,2-dimethoxy-butyronitrile were dissolved in 80 ml of absolute methanol and refluxed for 16 h. After distilling off the solvent under reduced pressure, the reaction solution was added a little at a time to a mixture of 18.4 g of KOH, 164 ml of ice water, and 40 ml of diethyl ether. The phases were separated and the aqueous phase was extracted with diethyl ether (5×10 ml). After drying the combined organic phases over NaSO₄ and KOH pellets, the solution was evaporated and could be converted directly into 2-propionyl-4,5-dihydro-1,3-thiazole.

Crude yield: 13.89 g (73.4 mmol, 91.7%).

¹H-NMR (400 MHz; CDCl₃): 0.85 (t, 3H, CH₃); 1.94 (q, 2H, CH₂); 2.27 (t, 8H, CH₂S and OCH₃); 4.38 (t, 2H, CH₂N)

### Example 2

### 2-Propionyl-4,5-dihydro-1,3-thiazole (I)—Individual Synthesis Steps

10.13 g (53.5 mmol) of 2-(1,1-dimethoxypropyl)thiazoline were added to 43 ml of sulfuric acid (96%) at 0 to 5° C. After stirring at this temperature for 20 min, the solution was added a little at a time to a mixture of 187 mg of NaHCO₃, 965 mg of ice, and 64 ml of diethyl ether. After phase separation, extraction of the aqueous phase with CH₂Cl₂, and drying of the combined organic phases over Na₂SO₄, the solvent was removed under reduced pressure.

The residue was distilled using a Vigreux column to give 3.099 g (21.6 mmol, 40% yield) of product having a purity of 98% according to gas chromatography (GC).

¹H-NMR (400 MHz; CDCl₃): 1.14 (t, 3H, CH₃); 2.95 (q, 2H, CH₂); 3.33 (t, 2H, CH₂S); 4.52 (t, 2H, CH₂N).

### Example 3

### Single-vessel Synthesis of 2-propionyl-4,5-dihydro-1,3-thiazole (I)—According to the Invention

536 mg of 1,1,1-trimethoxypropane (4 mmol), 0.53 ml of trimethylsilyl cyanide (4 mmol), and 1 mg of ZnCl₂ were heated at 60° C. under argon for 16 h. 339 mg of cysteamine (4.4 mmol), 154.2 mg of ammonium acetate (2.0 mmol), and 4 ml of methanol were added and the mixture was refluxed for a further 17 h. After removing the solvent under reduced pressure, 2.043 g of sulfuric acid (96%) were added dropwise at 0 to 5° C. After stirring at this temperature for 2 h, the reaction solution was added a little at a time to a mixture of 4.7 g of NaHCO₃ (56 mmol), 75 ml of ice water, and 5 ml of diethyl ether. The aqueous phase was extracted with CH₂Cl₂, after which the combined organic phases were dried over NaSO₄ and evaporated under reduced pressure.

Yield (crude product): 229 mg (40%); purity according to GC: 84%.

What is claimed is:

1. A process for preparing 4,5-dihydro-1,3-thiazoles of formula (I) 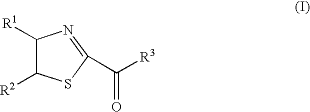
where R¹, R², and R³ are each, independently of one another,
hydrogen or an organic radical having from 1 to 10 carbon atoms, comprising
(1) reacting a trialkoxyalkane of the formula 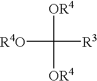
where R³ and R⁴ are each, independently of one another, hydrogen or an organic radical having from 1 to 10 carbon atoms,
with CN⁻ to form a 2,2-dialkoxyalkanenitrile of the formula 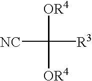
where R³ and R⁴ are defined as above,
(2) reacting the 2,2-dialkoxyalkanenitrile with an aminoalkanethiol of the formula 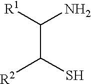
where R¹ and R² are defined as above,
to form a ketal of the formula 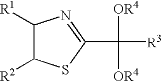
where R¹, R², R³, and R⁴ are defined as above, and
(3) hydrolyzing the ketal with an acid to form the 4,5-dihydro-1,3-thiazole of formula (I),
wherein the entire reaction sequence is carried out in a single vessel synthesis without isolation of intermediates.

2. A process according to claim 1 wherein R³ is ethyl.

3. A process according to claim 1 wherein CN⁻ is from trimethylsilyl cyanide.

4. A process according to claim 1 wherein the reaction of the trialkoxyalkane with CN⁻ is carried out in the presence of a catalytic amount of a Lewis acid.

5. A process according to claim 1 wherein equimolar amounts of trialkoxyalkane and cyanide are heated at a temperature of 40 to 100° C. in the presence of a catalytic amount of a Lewis acid.

6. A process according to claim 4 wherein the Lewis acid is ZnCl₂.

7. A process according to claim 1 wherein in step (2) from 1.0 to 1.5 equivalents of the aminoalkanethiol in an organic solvent are added to the dialkoxyalkanenitrile.

8. A process according to claim 7 wherein the organic solvent is distilled off under reduced pressure before step (3).

9. A process according to claim 1 wherein the acid used in step (3) is concentrated sulfuric acid.

10. A process according to claim 1 wherein in step (3) from 5 to 30 equivalents of the acid are added dropwise at a temperature of from 10° C. to −10° C.

11. A process according to claim 1 wherein after completion of step (3) the acid is neutralized with an aqueous base.

12. A process according to claim 1 additionally comprising extracting the 4,5-dihydro-1,3-thiazole of formula (I) into an organic phase and isolating the 4,5-dihydro-1,3-thiazole of formula (I).
